(19) United States
(12) Patent Application Publication
(10) Pub. No.: US 2012/0107932 A1
(43) Pub. Date: May 3, 2012

(54) PRRS-VIRUS RECEPTOR AND ITS INHIBITOR

(76) Inventor: Enmin Zhou, Shandong (CN)

(21) Appl. No.: 13/138,341

(22) PCT Filed: Apr. 16, 2009
(86) PCT No.: PCT/CN2009/071319

§ 371 Date: Nov. 23, 2011

(30) Foreign Application Priority Data
Feb. 4, 2009 (CN) 200910013683.3

(51) Int. Cl. C12N 5/02; C07K 7/08; C07K 16/00; C07K 7/06

(52) U.S. Cl. 435/375; 530/328; 530/327; 530/387.1

(57) ABSTRACT

This invention relates to a new cell receptor of the PRRS virus, i.e. as non-muscle myosin II-A (NMHC II-A)and its inhibitor blebbistatin, which can be used as a drug for suppressing PRRS virus infection of cells. The invention provides a method of utilizing purified NMHC II-A protein, artificially synthesized polypeptides and blebbistatin to prevent PRRS viruses from infecting cells. It also offers the antibodies generated by NMHC II-A protein and polypeptides. The purified NMHC II-A protein or artificially synthesized polypeptides and blebbistatin as well as anti-NMHC II-A protein and anti-polypeptide antibodies all have inhibitory effects on cell infection from the PRRS virus, and can be developed into drugs for preventing and treating infections of the PRRS virus.

TECHNICAL FIELD

This invention is about discovering a new PRRS-virus cell receptor as well as utilizing the receptor protein, polypeptides and the derivatives to prevent and treat infections from PRRS virus; therefore it is of the biological field.

### BACKGROUND TECHNOLOGY

Porcine reproductive and respiratory syndrome (PRRS), also called blue ear disease, is an acute porcine infectious disease caused by infection of the PRRS virus (PRRSV). After invading into host cells, PRRSV first combines with the specific receptor on the surface of the cell membrane. It then infects susceptible cells such as MA-104 and PAM by utilizing endocytosis (Nauwynck, H J, Duan X, Favoreel H W, etc. J. Gen. Virol. 1999, 80:297-305). To date, there are four reported PRRSV cell receptors that are independent of each other but have associated functions, including sialoadhesin (Vanderheijden N, Delputte P L, Favoreel H W, etc. J. Virol. 2003, 77:8207-8215), heparinlike (Delputte P L, Vanderheijden N, Nauwynck, H J, etc. J. Virol. 2002, 76:4312-4321), vimentin (Kim J K, Fahad A M, Shanmukhappa K, etc. J. Virol. 2006, 80:689-696) and CD163 protein (Calvert J G, Slade D E, Shields S L, etc. J. Virol. 2007. 81:7371-7379). However, there are also other PRRSV cell receptors, all of which can bind or participate in binding PRRSV-infected susceptible cells, thereby causing blue ear disease. Therefore, new viral receptors will be continuously discovered by studying PRRSV and thus the exploration scope can be extended.

### DISCLOSURE OF THE INVENTION

This invention relates to a new PRRSV cell receptor and meanwhile it provides a method to inhibit PRRSV from infecting cells by purified NMHC II-A proteins, artificially synthesized polypeptides and blebbistatin as receptor inhibitor; the antibodies obtained by immunizing mice with NMHC II-A proteins and polypeptides can also be used as receptor inhibitor. All the blockers above have inhibitory effects on cell infection caused by PRRSV, and can be developed into medicines for prevention and treatment of PRRSV infection.

The applicant first identified a soluble protein on MA-104 and porcine alveolar macrophages (PAM) by internal image monoclonal anti-idiotypic antibodies against idiotypic antibodies to GP5 antigen of PRRSV (Zhou E M, Xiao Y H, Shi Y F, etc. J. Virol. Methods. 2008, 149:300-308). This protein is determined as non-muscle myosin heavy chain II-A (NMHC II-A) by analyzing its sequence of amino acids; the amino-acid sequence of it at the carboxyl terminal is illustrated in Seq ID No:1.

The blockers and inhibitors of this receptor have also been found, including NMHC II-A protein, NMHC II-A polypeptides, anti-NMHCII-A protein and anti-polypeptide antibodies, and the inhibitor of non-muscle myosin II (NMM-II), i.e. (+)-blebbistatin (an inactive enantiomer of the NMM-II selective inhibitor [(±)- or (−)-blebbistatin]). Where:

(a) NMHC II-A protein, of which the amino-acid sequence at the carboxyl terminal is illustrated in Seq ID No: 1;

(b) NMHC II-A artificial polypeptide, of which the amino-acid sequence is illustrated in Seq ID No: 2;

(c) NMHC II-A artificial polypeptide, of which the amino-acid sequence is illustrated in Seq ID No: 3;

(d) NMHC II-A artificial polypeptide, of which the amino-acid sequence is illustrated in Seq ID No: 4.

In the viral receptors as well as their blockers and inhibitors above, the ones that target PRRSV include classic American strains (representative strain: VR-2332), Chinese isolates (representative strain: Ch-1a) and highly pathogenic strains (representative strain: JXA-1). And the inhibitor of non-muscle myosin II (NMM-II), i.e. (+)-blebbistatin, can react with NMM-II and block the classic American strains, Chinese isolates and highly pathogenic strains of PRRSV from infecting cells. Unlike (±)- or (−)-blebbistatin, (+)-blebbistatin does not inhibit the NMM-II-dependent cellular division and proliferation (Straight A F, Cheung A, Limouze J, etc. Science. 2003, 299:1743-1747). The (±)-blebbistatin is always a selective inhibitor of NMM-II ATP are activity on vertebrate cells; the inventor has used NMHC II-A proteins as a PRRSV receptor and meanwhile found that (+)-blebbistatins could be regarded as receptor blockers to prevent PRRSV from infecting cells after performing long-term tests, and this application has not been reported in previous studies.

The detailed isolation and identification procedures of this viral receptor, i.e. non-muscle myosin heavy chain II-A (NMHC II-A) protein, are as follows:

The soluble protein on MA-104 and porcine alveolar macrophages (PAM) were purified by internal image monoclonal anti-idiotypic antibodies against idiotypic antibodies to GP5 antigen of PRRSV (Zhou E M, Xiao Y H, Shi Y F, et al. J. Virol. Methods, 2008, 149:300-308); 50 μg of the soluble protein above was separated with 7.5% SDS-PAGE, hydrolyzed with pepsin, separate the hydrolyzed fragments by micro-capillary HPLC, perform nano-electrospray ionization to the isolated fragments, and then analyze with an ion-capturing mass spectrometer. The obtained receptor protein is determined as non-muscle myosin heavy chain II-A (NMHC II-A) after comparing the acquired hydrolyzed fragment with GenBank amino-acid sequence bank.

The detailed synthesis method for the (b), (c) or (d) above is as follows: According to the amino-acid sequence of the NMHC II-A heavy chain protein, analyze the epitope of this sequence by using DNA Star software and design 20 polypeptide sequences, then synthesize the polypeptides by the solid-phase polypeptide synthesis method, and couple the synthesized polypeptide with the carrier protein Keyhole Limpet Hemocyanin (KLH) by MBS or glutaraldehyde. Meanwhile, the PPRSV blocking test by using the artificially synthesized polypeptides above has demonstrated that the 23 amino acids at the carboxyl terminal of NMHC II-A is the binding region of PRRSV; therefore, the carboxyl terminal has the same protein as Seq ID No: 1 in the sequence list, which can be used as the viral receptor and the corresponding blocker.

For the anti-NMHC II-A protein and anti-polypeptide antibodies generated by mixing NMHC II-A protein and polypeptide-KLH coupling product with aluminum adjuvant to immunize mice, the serum antibodies can be obtained by abdominal immunization of mice as well as other antibody preparation methods. For example, they can be prepared by mixing with other adjuvants and performing intramuscular or subcutaneous immunization. The specific immunization method depends on the availability of immunization methods in current biology.

In general, the inventor has used the protein with the same carboxyl terminal of Seq ID No: 1 in the sequence list, especially the non-muscle myosin heavy chain II-A (NMHC II-A) protein, as a PRRSV receptor, therefore all the relevant materials above have inhibitory activity to cell infection from PRRSV, and can be developed into drugs for preventing and treating PRRSV infection. Thus, this provides a brand-new idea for PRRS studies as well as its prevention and treatment, extends the exploration scope, and has the utmost significance to actual production.

### PREFERRED EMBODIMENTS

### Example 1

### Discovery And Identification of A New PRRSV Cell Receptor

The soluble protein on MA-104 and porcine alveolar macrophages (PAM) were purified by Soluble protein from MA-104 and PAM cells were purified by internal image monoclonal anti-idiotypic antibodies against idiotypic antibodies to GP5 antigen of PRRSV (Zhou E M, Xiao Y H, Shi Y F, et al. J. Virol. Methods, 2008, 149:300-308); 50 μg of the soluble protein above was separated with 7.5% SDS-PAGE, hydrolyzed with pepsin, separate the hydrolyzed fragments by micro-capillary HPLC, perform nano-electrospray ionization to the isolated fragments, and then analyze with an ion-capturing mass spectrometer. The obtained receptor protein is determined as non-muscle myosin heavy chain II-A (NMHC II-A) after comparing the acquired hydrolyzed fragment with GenBank amino-acid sequence bank.

### Example 2

### Artificial Synthesis of NMHC II-A Polypeptides

The polypeptides are synthesized with the solid-phase polypeptide synthesis method as per the polypeptide sequence determined by the carboxyl terminal of the NMHC II-A heavy chain, and the detailed procedure is as follows:

Mix the 2-Chlorotrityl Chloride Resin with DMF and shake 30-60 minutes to swell the resin; remove the DMF by suction filtration with a sand core, add excessive three-fold mole of the first amino-acid at the C-terminal to make them attach to the resin, then add excessive ten-fold mole of DIEA and finally add the DMF to dissolve them, shake 30-60 minutes; remove the DMF, add 20% piperidine in the DMF, and react 5-15 minutes; get dozens of resin particles, wash three times with alcohol, then add a drop of ninydrin, KCN and phenol solutions respectively, and heat five minutes at 105□-110□ to test the reactant. Wash twice with DMF, methanol and DMF respectively in turn. Condense the attached products by one of the following methods.

Method a: Add three-fold excess of the protective amino acid in DMF (FOMC-Asp-OH) and HBTU, then add excessive ten-fold NMM, and react 30-60 minutes.

Method b: Add three-fold excess of the protective amino acid in DMF (FOMC-Asp-OH) and HOBt, then add excessive three-fold DIC, and react 30-60 minutes.

After that, wash twice with DMF, methanol and DMF respectively in turn. Repeat the steps above to attach all the amino acids in the sequence successively. After attachment of the final amino acid, wash twice with DMF, methanol, DMF and DCM respectively in turn, and dry by suction for 10-20 minutes. Cut 120 minutes with the cutting solution (TFA 94.5%, water 2.5%, EDT 2.5%, TIS 1%) to separate the polypeptide from the resin, blow with nitrogen to make it dry, wash six times with ether, and volatilize-dry at normal temperature. Dissolve the rough polypeptide with pure water or a small quantity of acetonitrile, and purify the polypeptide by HPLC as per the following conditions. The chromatographic column is Venusi MRC-ODS C18, 30×250 mm; the solution in Pump A is 0.1% trifluoroacetic in 100% pure water; the solution in Pump B is 0.1% trifluoroacetic in 100% acetonitrile. Finally, lyophilize the purified polypeptide and store at −20□.

Alternatively, the polypeptides can be synthesized by other methods, for example the solid-phase synthesis method by using tert-butoxy-carbonyl or 9-fluorenylmethoxycarbonyl (Merrifield R B. J. AM. Chem. Soc., 1963, 85:2149-2154; Chang C D and Merenhofer J. Int. J. Pept. Protein Res. 1978, 11:246-249).

### Example 3

### Coupling Polypeptide With the Carrier Protein Keyhole Limpet Hemocyanin (KLH)

Each polypeptide is coupled with KLH by one of the following methods:

(1) Coupling with a coupler MBS (M-Maleimidobenzoyl-N-hydroxysuccinimide): Dissolve 5 mg KLH with 1 mL PBS (0.1M, pH 6.0), and add 50 μL DMSO (containing 1 mg MBS) to react an hour; separate the reaction products by HPLC and add 5 mg of the polypeptide to react three hours, and finally perform vacuum lyophilization.

(2) Glutaraldehyde coupling: Dissolve 5 mg KLH in deionized water, add a certain amount of glutaraldehyde and 5 mg of the polypeptide, and then react five to six hours; dialyze 24 hours with sodium bicarbonate and sodium carbonate buffer solutions, and finally perform vacuum lyophilization.

### Example 4

### Preparation of Anti-NMHC II-A Protein And Artificial Anti-Polypeptide Antibodies

Preparation of the protein and polypeptide-aluminum adjuvant: Dissolve the NMHC II-A protein or polypeptide-KLH coupling product in 0.005M PBS (pH 6.2, final volume 10 mL), add slowly aluminum potassium sulfate in 0.005M PBS (pH 6.2) (ratio: 50 μg protein/0.4 mg aluminum), and elevate the pH to 6.8-7.3 and stir two hours; centrifuge 800 g for ten minutes, then wash the protein-aluminum adjuvant precipitate once with saline, and finally dissolve it with 0.1M PBS. The protein and polypeptides can also be used with other adjuvants such as Freund's Adjuvant.

Immunization procedure: Abdominally immunize each mouse with 50 μg NMHC II-A protein or an artificial polypeptide, perform co-immunization three times, and collect the mouse serum for antibody testing and identification. The anti-NMHC II-A protein or artificial anti-polypeptide antibodies can also be used to immunize other animals (e.g. rabbits, goats, etc.), and other immune routes (such as intramuscular or subcutaneous) can be adopted. The spleen of the mouse generating antibodies can also be utilized to prepare monoclonal antibodies by cell fusion.

### Example 5

### Identification of NMHC II-A Protein, Artificially Synthesized Polypeptides And Their Antibodies

Indirect ELISA method: First, envelop the NMHC II-A protein and all the polypeptides with PBS (0.01 M, pH 7.2) onto an ELISA plate, add 200 ng for each well, and place overnight at 4□. Wash the ELISA plate with PBS-T (PBS containing 0.5% volume of Tween-20) three times and then seal for an hour with the confining liquid (2.5% concentration of skim milk powder in PBS-T, 200 μl/well), and wash three times; add the diluted mouse serum into each well, let it react an hour, and then wash three times; add the goat anti-mouse IgG labeled with horseradish peroxidase (HRP) into each well, react an hour, and then wash three times; add 100 μl HRP substrate (TMB) into each well and react 15 minutes; add 50 μl 1M sulphuric acid into each well for reaction termination, and read the OD value of each well by an ELISA analyzer at 450 nm. The test results are presented in Tables 1 and 2. The mouse serum before immunization is used as negative control serum; and if the OD value from the immunized mouse serum is three times higher than that of negative control, the result is positive. As demonstrated by the results in Table 1, NMHC II-A protein is of immunogenicity, the serum antibody has a binding feature with NMHC II-A protein and the antibody titre can reach 1:100000. Meanwhile, these serum antibodies can combine with artificially synthesized polypeptides and the antibody titre can reach 1:10000. As demonstrated by the results in Table 2, the mice immunized by the three artificially synthesized polypeptides can bind not only the polypeptides for immunization use but also the NMHC II-A protein; and the antibody titre can reach 1:10000.

TABLE 1
Results of Anti-NMHC II-A Serum Antibodies
Reacting with the Antigens

| | OD values of serum-bound antigen under different | | | | | | | |
|---|---|---|---|---|---|---|---|---|
| | dilutions | | | | | | | |
| | | | | | Sera after third | | | |
| | Negative control sera | | | | immunization | | | |
| Antigen | 10⁻² | 10⁻³ | 10⁻⁴ | 10⁻⁵ | 10⁻² | 10⁻³ | 10⁻⁴ | 10⁻⁵ |
| NMHC II-A | 0.23 | 0.20 | 0.15 | 0.09 | 3.27 | 2.16 | 1.08 | 0.46 |
| protein | | | | | | | | |
| Polypeptide | 0.15 | 0.14 | 0.11 | 0.10 | 0.78 | 0.53 | 0.38 | 0.16 |
| (Seq ID No: 2) | | | | | | | | |
| Polypeptide | 0.17 | 0.16 | 0.13 | 0.11 | 0.69 | 0.48 | 0.33 | 0.11 |
| (Seq ID No: 3) | | | | | | | | |
| Polypeptide | 0.16 | 0.12 | 0.10 | 0.08 | 0.75 | 0.51 | 0.39 | 0.17 |
| (Seq ID No: 4) | | | | | | | | |

TABLE 2

| | OD values of serum-bound antigen under different | | | | | | | |
|---|---|---|---|---|---|---|---|---|
| | dilutions | | | | | | | |
| | | | | | Serum after third | | | |
| | Negative control sera | | | | immunization | | | |
| Antigen | 10⁻² | 10⁻³ | 10⁻⁴ | 10⁻⁵ | 10⁻² | 10⁻³ | 10⁻⁴ | 10⁻⁵ |

Results of Anti-Polypeptide (Seq ID No: 2) Serum
Antibodies Reacting with the Antigens

| | | | | | | | | |
|---|---|---|---|---|---|---|---|---|
| Polypeptide | 0.12 | 0.11 | 0.08 | 0.07 | 1.25 | 0.78 | 0.41 | 0.25 |
| (Seq ID No: 2) | | | | | | | | |
| NMHC II-A | 0.13 | 0.10 | 0.10 | 0.08 | 1.17 | 0.68 | 0.35 | 0.19 |
| protein | | | | | | | | |

Results of Anti-Polypeptide (Seq ID No: 3) Serum
Antibodies Reacting with the Antigens

| | | | | | | | | |
|---|---|---|---|---|---|---|---|---|
| Polypeptide | 0.11 | 0.13 | 0.09 | 0.08 | 1.16 | 0.75 | 0.43 | 0.22 |
| (Seq ID No: 3) | | | | | | | | |
| NMHC II-A | 0.12 | 0.11 | 0.8 | 0.07 | 1.05 | 0.65 | 0.32 | 0.17 |
| protein | | | | | | | | |

Results of Anti-Polypeptide (Seq ID No: 4) Serum
Antibodies Reacting with the Antigens

| | | | | | | | | |
|---|---|---|---|---|---|---|---|---|
| Polypeptide | 0.15 | 0.13 | 0.10 | 0.09 | 1.20 | 0.82 | 0.47 | 0.28 |
| (Seq ID No: 4) | | | | | | | | |
| NMHC II-A | 0.14 | 0.11 | 0.08 | 0.07 | 1.11 | 0.72 | 0.38 | 0.23 |
| protein | | | | | | | | |

Indirect immunofluorescence assay (IFA): After dilution, combine the mouse anti-NMHC II-A protein and anti-polypeptide antibodies after immunization three times with MA-104 and PAM monolayer cells for 0.5-2 hours, then bind the goat anti-mouse IgG testing antibody labeled with fluorescence to the cells. The test results are presented in Table 3. As demonstrated by the results in Table 3, the serum antibodies have the cell-bound feature and the antibody titre can reach 1:320.

TABLE 3
Results of Indirect Immunofluorescence Assay for Detecting
Serum Antibodies Binding to MA-104 and PAM Cells

| | Cell-bound serum under different dilutions (with or without | | | | | | | |
|---|---|---|---|---|---|---|---|---|
| | fluorescence) | | | | | | | |
| | | | | | Serum after third | | | |
| | Negative control sera | | | | immunization | | | |
| Antigen | 1/40 | 1/80 | 1/160 | 1/320 | 1/40 | 1/80 | 1/160 | 1/320 |
| NMHC II-A | No | No | No | No | Yes | Yes | Yes | Yes |
| protein | | | | | | | | |
| Polypeptide | No | No | No | No | Yes | Yes | Yes | Yes |
| (Seq ID | | | | | | | | |
| No: 2) | | | | | | | | |
| Polypeptide | No | No | No | No | Yes | Yes | Yes | Yes |
| (Seq ID | | | | | | | | |
| No: 3) | | | | | | | | |
| Polypeptide | No | No | No | No | Yes | Yes | Yes | Yes |
| (Seq ID | | | | | | | | |
| No: 4) | | | | | | | | |

### Example 6

### Effects of NMM-II Inhibitor (Blebbistatin) On Cells

(±)-blebbistatin is a selective inhibitor of NMM-II ATPase activity on vertebrate cells; it can inhibit the contraction of cellular cleavage groove, rapidly and reversibly block cells from bubbling, and prevent cell migration and cytokinesis (Straight A F, Cheung A, Limouze J, etc. Science. 2003, 299:1743-1747). (−)-blebbistatin is the active enantiomer of (±)-blebbistatin, and (+)-blebbistatin is the inactive enantiomer of (±)-blebbistatin. Marc-145 and PAM cells (10⁵ cells/ml) are cultured on DMEM medium (containing 100 U/ml penicillin, 50 μg/ml streptomycin sulphate, 50 μg/ml gentamycin and 10% calf serum) in an incubator under 37□ and 5% CO₂ to form monolayer. Dissolve (±)-blebbistatin, (S)-(−)-blebbistatin and (R)-(+)-blebbistatin (purchased from Tocris Cookson, Inc. [US]) in DMSO, then perform multiple-proportion dilution with PBS, add a monolayer culture plate and continuously culture it to observe cytopathic effects (CPE). Under a conventional microscope, CPE is observed as rough cell surface, strong refractivity, and finally cell shedding. The results in Table 4 show that (±)-blebbistatin and (S)-(−)-blebbistatin can inhibit cell proliferation and cause CPE. However, (R)-(+)-blebbistatin does not inhibit cell proliferation and does not cause CPE. Therefore, (+)-blebbistatin can be used as a PRRSV-receptor blocker.

TABLE 4
Effects of Blebbistatin on MA-104 and PAM Cells

| | Different concentrations (μM) of | | | | | | |
|---|---|---|---|---|---|---|---|
| | blebbistatin causing CPE or not (Yes or No) | | | | | | |
| Blebbistatin | 100 | 50 | 20 | 10 | 5 | 2 | 1 |
| (R)-(+)- | No | No | No | No | No | No | No |
| blebbi- | | | | | | | |
| statin | | | | | | | |
| (±)- | Yes | Yes | Yes | Yes | Yes | Yes | Yes |
| blebbi- | | | | | | | |
| statin | | | | | | | |
| (S)-(−)- | Yes | Yes | Yes | Yes | Yes | Yes | Yes |
| blebbi- | | | | | | | |
| statin | | | | | | | |

### Example 7

### PRRSV-Receptor Blocking Test

Marc-145 and PAM cells (10⁵ cells/ml) are cultured on DMEM medium (containing 100 U/ml penicillin, 50 μg/ml streptomycin sulphate, 50 μg/ml gentamycin and 10% calf serum) in an incubator under 37 □ and 5% CO₂ to form monolayer. Dissolve the NMHC II-A protein, artificially synthesized polypeptides, serum antibodies and (+)-blebbistatin obtained by using the methods of Implementation Examples 1, 2 and 4 in the DMEM medium; after de-germing by filtration, mix with PRRSV strains under different fixation concentrations (10³TCID₅₀/ml) and incubate an hour, and then add the cells above; perform eight repetitions for each dilution; after adsorption at 37□ for an hour, remove the mixture, add DMEM medium and culture continuously; and observe CPE daily. The CPE manifestations caused by PRRSV infection include rough cell surface, strong refractivity, and finally cell shedding. The results in Table 5 illustrate the CPE inhibition levels of the NMHC II-A protein, artificially synthesized polypeptides, serum antibodies and (+)-blebbistatin under the each-well concentration of μg, μg, μL and μM in turn. The judgment results of cell CPE are expressed as follows: “−” means no CPE, “+” means CPE in 25% cells, “++” means CPE in 50% cells, and “+++” means CPE in above 75% cells. The results in Table 5 show that NMHC II-A protein and artificially synthesized polypeptides (Seq ID Nos. 2, 3 and 4) at 2 μg-100 μg/well, anti-NMHC II-A protein and artificially synthesized anti-polypeptide serum at 5 μL-100 μL/well, and (+)-blebbistatin at 2 μM-100 μM/well can fully inhibit the cell CPE caused by PRRSV; this demonstrates that NMHC II-A protein and its derivatives can block PRRSV from infecting cells, and can be used to develop drugs for prevention and treatment of PRRSV.

TABLE 5
Results of PRRSV-Receptor Blocking Test

| | Inhibition levels of CPE under | | | | | | |
|---|---|---|---|---|---|---|---|
| | different concentrations | | | | | | |
| Receptor blocker | 100 | 50 | 20 | 10 | 5 | 2 | 1 |
| NMHC II-A protein | − | − | − | − | − | − | + |
| Polypeptide (Seq ID No: 2) | − | − | − | − | − | − | ++ |
| Polypeptide (Seq ID No: 3) | − | − | − | − | − | − | ++ |
| Polypeptide (Seq ID No: 4) | − | − | − | − | − | − | ++ |
| Anti-NMHC II-A protein | − | − | − | − | − | + | +++ |
| serum | | | | | | | |
| Anti-polypeptide (Seq ID | − | − | − | − | − | + | +++ |
| No: 2) serum | | | | | | | |
| Anti-polypeptide (Seq ID | − | − | − | − | − | + | +++ |
| No: 3) serum | | | | | | | |
| Anti-polypeptide (Seq ID | − | − | − | − | − | + | +++ |
| No: 4) serum | | | | | | | |
| (+)-blebbistatin | − | − | − | − | − | − | + |

1. A receptor blocker that can block the PRRS virus from infecting cells, which is selected from the blockers with one of the following features:
(a) An artificial polypeptide, of which the amino-acid sequence is illustrated in Seq ID No: 2;
(b) An artificial polypeptide, of which the amino-acid sequence is illustrated in Seq ID No: 3;
(c) An artificial polypeptide, of which the amino-acid sequence is illustrated in Seq ID No: 4;
(d) Antibody prepared by using (a), (b) or (c).

2. The use of a NMHC II-A protein, which is described as a receptor blocker to prevent the PRRS virus from infecting cells.

3. The use of a non-muscle myosin II (NMM) inhibitor, i.e. (+)-blebbistatin, which is described as a receptor blocker to prevent the PRRS virus from infecting cells.

4. The use of the antibody prepared with the NMHC II-A protein, which is described as a receptor blocker to prevent the PRRS virus from infecting cells.

5. The receptor blockers for blocking the PRRS virus from infecting cells according to claim 1, characterized, characterized in that, they are to bind the PRRS virus or cell receptor and block the PRRS virus from infecting MA-104 and PAM.

6. The receptor blockers for blocking the PRRS virus from infecting cells according to claim 1, characterized, characterized in that, it will be used to develop drugs for prevention and treatment of PRRS diseases.


SEQUENCE LISTING (4 sequences)

SEQ ID NO: 1 - protein, 23 aa, Porcine Reproductive and Respiratory Syndrome Virus
GAGDGSDEEVDGKADGAEAKPAE

SEQ ID NO: 2 - protein, 9 aa, Artificial Sequence
  Description of Artificial Sequence Synthetic polypeptide
GAGDGSDEE

SEQ ID NO: 3 - protein, 14 aa, Artificial Sequence
  Description of Artificial Sequence Synthetic polypeptide
GAGDGSDEEVDGKA

SEQ ID NO: 4 - protein, 11 aa, Artificial Sequence
  Description of Artificial Sequence Synthetic polypeptide
KADGAEAKPAE